(19) United States
(12) Patent Application Publication
(10) Pub. No.: US 2014/0343450 A1
(43) Pub. Date: Nov. 20, 2014

(54) Cognitive testing for drug compliance monitoring

(71) Applicant: Sync-Think, Inc., Boston, MA (US)

(72) Inventor: Matthew E. Stack, Pittsfield, MA (US)

(73) Assignee: Sync-Think, Inc., Boston, MA (US)

(21) Appl. No.: 13/986,617

(22) Filed: May 16, 2013

(51) Int. Cl. A61B 5/00; A61B 3/113

(52) U.S. Cl. CPC A61B 5/4833; A61B 3/113; A61B 5/6803
USPC 600/558; 600/300

(57) ABSTRACT

A system for monitoring drug taking compliance utilizes cognitive testing after the scheduled administration of the medicine to determine compliance by measuring cognitive function.

### FIELD OF THE INVENTION

This invention relates to cognitive testing and more particularly the utilization of cognitive testing for drug taking compliance.

### BACKGROUND OF THE INVENTION

Drug compliance has always been a challenge to the pharmaceutical and biotech industries that invest heavily in creating drugs to be administered to patients on a regime and a therapeutic program. Therapeutic regimes or the programs developed for the patients are highly structured and optimized in order to maximize the efficiency of drug or compound delivery to the body. In order to treat the medical condition or ailment that the compound or drug is taken for, many of these regimes require weekly, daily, and sometimes intra-daily repeated doses to remain the most efficacious. The amount of drug or compound in the human body system is a tightly controlled system where the natural metabolism seeks to eliminate the drug while the pharmaceutical company theory of intervention in the program built to administer the drug over a period of time seeks to maximize the amount of bioavailable compound in the blood stream and in the body system. This repeat dosage and frequent repeat dosing is part of the recurring challenge of taking certain classes of pharmaceutical products, specifically those that are oncological for cancer purposes or dementia related or involve other chronic diseases that develop over time or require mitigation over time.

It is worth noting that there is a class of drugs that are specifically designed to treat dementia which are meant to be taken on a regular or recurring basis and that are specifically administered or provided to individuals that suffer from other cognitive ailments including lapse of memory or impaired memory. Thus, the problem of drug compliance is heightened with the patient population because these are patients who are less likely to remember to take the drugs that need to be taken daily if not multiple times a day.

The same can also be said for a class of drugs that pertains to cognitive disorders such as attention deficit disorders, epilepsy or Parkinson's disease. These drugs are used to treat a state of mind and the state of mind has a high correlation with other impairments such as will or memory. Thus, this is a problem because cognitive disorder patients tend to exhibit behavioral patterns that amplify drug non-compliance.

A whole industry has sprung up within the medical products and medical technologies sector of the economy to service the drug compliance problem. This is because the economics of drug compliance are quite obvious to those practitioners but are recapped briefly here. From a pharmaceutical perspective, drug compliance means greater sales of the drugs. Moreover, it leads to healthier patients due to higher efficacy and due to increased impact results for the pharmaceutical product taken.

In addition, the risks to the insurance companies for not providing recurring and repeated treatment programs is high because the insurance companies will frequently be faced with higher costs in the wake of patient non-compliance or because the patient did not take the medication to treat the ailment they had. In general, insurance companies are at risk when for behavioral ailments patients take pharmaceuticals. This is because non-compliance might lead to more severe injuries, bodily harm, or other damages or risks.

The spectrum of technologies employed to service the monitoring of patients compliance with their drug regimes is diverse. On one end there are simple low technology solutions which involve pill reminders or pill bottle containers that have calendar systems to encourage patients to remember what day it is and whether they have taken their medication for that day or not. On the higher end, and employing more diverse technologies, there are electronic systems and cell phone and mobile device applications that help remind a patient in the form of recurring alarms. Further still are business model innovations for instance such as social media that impact social stigma suggesting that medication has not been taken on time or in accordance to a program. There are also business models such as telemedicine, which encourage the use of exterior monitoring and third party monitoring as to whether the patient is being compliant with their program.

Current technology invokes simple reminder systems. With few exceptions when using the simple systems it is difficult to get an instantaneous and recurring measurement as to whether or not the patient is taking their medication. The only exception is a reminder or a check-in system that ascertains if medication has been taken by the patient. Nonetheless it is difficult to determine whether the patient is just asserting they have taken it or if they have really taken it. Moreover, this requires the patients to submit answers to a survey to ascertain the reality of whether the patient is taking the medication or not.

In a rare few instances there exist alignment of medical monitoring, drug compliance, and disease state. For instance for diabetes where blood sugar monitoring is relatively straight forward and difficult to fake, diabetes medication has such a linear implication on blood sugar that medication compliance is straight forward. Thus, blood glucose levels monitoring technology is nicely aligned with the disease state. As a result in some limited circumstances there is technology that can be put into place to monitor the patient to monitor whether or not the patient has taken the medicine. For instance monitoring of blood glucose level instead of filling out a survey is a better way of ascertaining patient compliance.

On the other hand, for oncology or cognitive impairments, these disease states take much longer to manifest change for failure to take medicine. As a result the easiest way to remotely monitor whether an epileptic person is taking their medicine or whether a Parkinson's disease patient is taking Parkinson's disease medication is to simply ask how many pills they have taken or to measure how many pills the patient has taken. However pill counting can be confounded by the fact that the pills can be thrown out instead of ingested. In the case of oncology diagnostics that can be deployed to the home that measure whether tumors have shrunken or grown, these diagnostic techniques are often very difficult to administer and are only useful when a visible tumor is superficially manifested. Thus, tumor response can be measured with simple technology such as a ruler or a camera and picture used to measure tumor growth

### SUMMARY OF THE INVENTION

Cognitive testing modalities that involve opto-cognitive scanning, which assesses a patient's eye-to-brain interface through quantification of smooth pursuit eye movement scores in a static or adaptive feedback driven system, provide a statistically accurate test of medication compliance due to the fine tuned quantitative score of cognitive function available with smooth pursuit eye movement testing. This score and process can then be used to monitor patient compliance by monitoring short-term effects of not taking the prescribed medicine.

To this end, adaptive static and hybrid smooth pursuit eye movement is an example of a test quantifiably rigorous enough to be useful in the measurement of whether or not a patient is taking his medicine.

For instance, taking medication such as progesterone for mTBI, or L-dopa for Parkinson's disease or any other host of drugs for the treatment of dementia can be monitored by the smooth pursuit eye movement test.

This can be accomplished in practice for instance, by asking patients to check-in at the beginning of each day to take an opto-cognitive test and or other cognitive tests, or for instance at the end of the day or some mid point of the day, or for instance shortly after having taken their medication. This serves as a stamp or check of patient compliance in that is difficult to fake. Smooth pursuit testing also provides very transparent quantitative scoring of whether a patient has taken their medication on a day-to-day basis.

On back end or processing side from a clinic or physicians office, the physicians see whether or not the smooth pursuit scores are improving or remaining the same or not. Thus, the physician can make a clinical determination about whether the patient is in fact compliant with their medication.

This only works if the test has a quantitative high resolution, making it difficult to fake the test results. Otherwise it would be difficult to determine the outcome of the test, let alone the validity the data collected.

The process assumes that the technology cost for providing one of these devices is low enough, effective enough, and hard and rugged enough that it can survive the stresses of being placed in a patient's home. This in turn relies on the existence of rugged structural design, consumer design, and user friendliness to make opto-cognitive testing available in a home environment and in a way that patients do not mind taking the test. Assuming these can be met with an appropriately handled device, the recurring cognitive testing through opto-cognitive testing paradigms and tests permits one for the first time to get a reliable measurement on a repeated basis on whether the patients are in fact complying with their medical regime and drug program and taking the appropriate and necessary drug treatments.

The application of opto-cognitive testing on a relatively low cost device placed at a patients home is vastly superior and cheaper in many ways across the healthcare value chain as compared to patient check-ups by medical staff or through registered nurse visits; or through other forms of periodic check-ins that require human intervention and manual labor. Since the test is capable of self-administration, it also does not require clinical supervision in order to get a daily check-in. It also permits one to get a much more frequent examination in home environments than any other previous technologies. The result is a repeated reliable consistent measure of data of drug compliance over a period of time in home or non-hospital environments.

### BRIEF DESCRIPTION OF THE DRAWINGS

These and other features of the subject invention will be better understood in connection with the detailed description in conjunction with the drawings of which:

FIG. 1 is a diagrammatic illustration of an individual taking prescribed medication;

FIG. 2 is a diagrammatic illustration of the use of opto-cognitive testing in the form of a smooth pursuit eye-tracking test, the output of which is used to ascertain drug compliance;

FIG. 3 is a diagrammatic illustration of advanced opto-cognitive test where a finger is to follow a moving target and a headset mounted opto-cognitive eye tracking unit for measuring cognitive function; and,

FIG. 4 is a flow chart showing one algorithm for measuring cognitive function using the desktop system.

### DETAILED DESCRIPTION

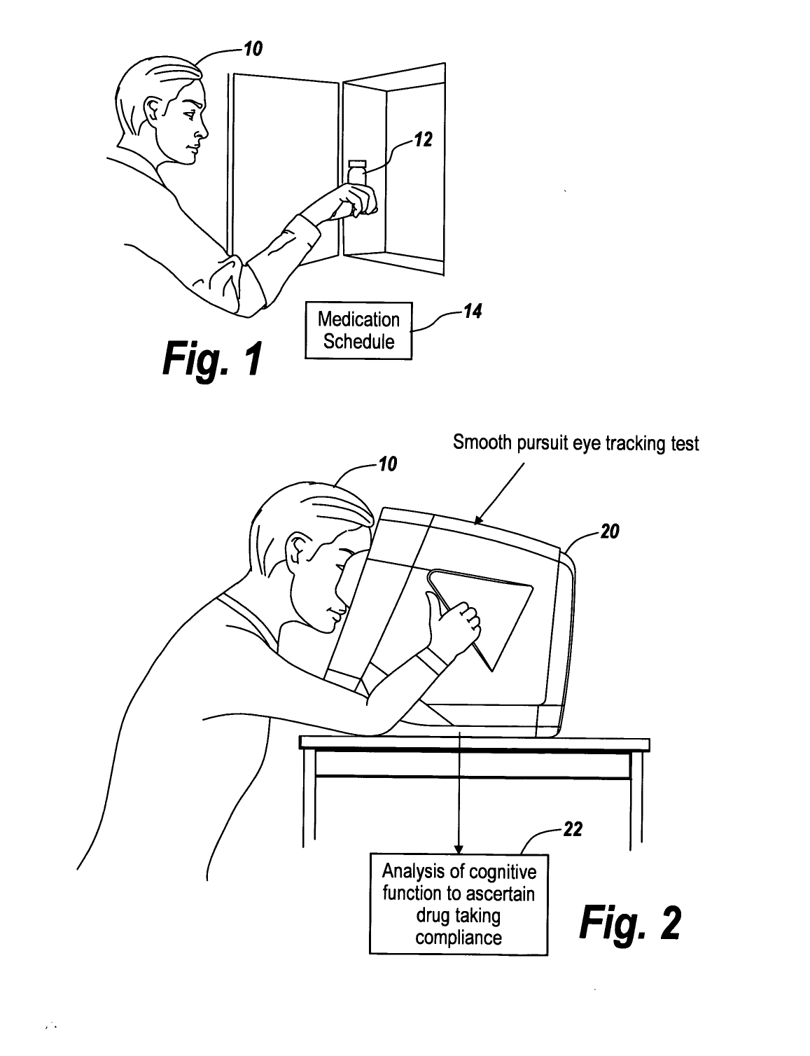

As illustrated in FIG. 1, an individual 10 is instructed by his or her physician to take a medication 12 in accordance with a medication schedule 14 prescribed by the physician.

It will be appreciated that often times the patients do not take medications on schedule or forget to take the medication completely.

The questioning of the patient as to compliance with the medication schedule is not very useful either because of the patient's inability to remember whether or not he or she took a particular medication or because the patient is in fact not telling the truth. Thus the problem of ascertaining drug taking compliance is often times difficult.

In order to solve the problem, as illustrated in FIG. 2, individual 10 is provided with opto-cognitive testing apparatus 20 in which for instance a smooth pursuit eye-tracking test is performed. In one embodiment this particular smooth pursuit eye tracking test apparatus is a desktop unit more fully described in U.S. patent application Ser. No. 13/507,991 filed Aug. 10, 2012 and incorporated herein by reference. While the subject invention will be described in connection with the desktop model, there are other methods of performing opto-cognitive testing, namely the headset device such as described in U.S. patent application Ser. No. 13/506,870 filed Mar. 11, 2012 and incorporated herein by reference. Also, as illustrated in U.S. patent application Ser. No. 12/931,881 filed Feb. 12, 2011 a manual method of utilizing the eyes to track a moving object on a screen can be used to ascertain cognitive performance. In this patent a tablet presents an on-screen target which is moved in a smooth pursuit path that is followed by the individual's finger and thus involves opto-cognitive testing.

As more fully described in the above-mentioned patent applications a target is moved along a smooth pursuit eye-tracking path, with the motion of the person's eyes and thus the person's gaze being the recorded measurement parameter. As the target moves, the person's gaze direction changes to follow the target and this gaze direction is used as a measure of cognitive function.

As described in U.S. patent application Ser. Nos. 13/694,462; 13/694,873, 13,694,461 and 13/815,574, all incorporated by reference, various techniques utilizing smooth pursuit eye tracking have been found to be effective and reliably consistent measures of cognitive ability.

Having measured cognitive performance by any opto-cognitive testing mechanism, as shown at 22 this opto-cognitive test is utilized to analyze the cognitive function of the individual and thus to ascertain drug taking compliance.

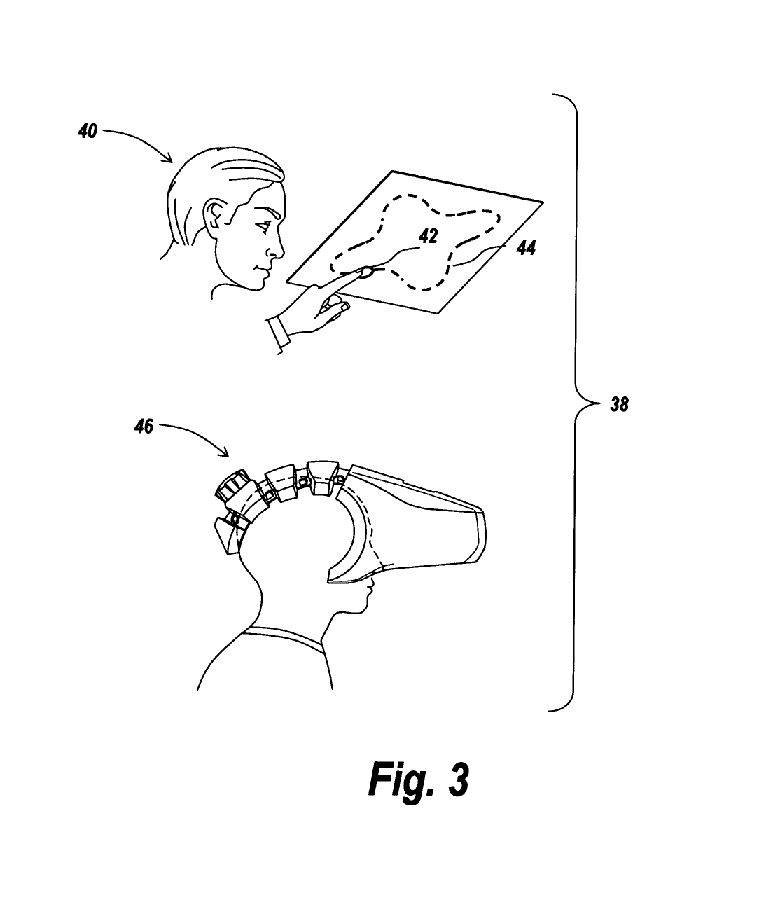

As illustrated in FIG. 3, these and other systems 38 that can be used for measuring cognitive performance. As shown at 40 a person's cognitive performance can be measured by moving his finger to track a target 42 moving along a smooth pursuit path 44. Alternatively, and as described in U.S. patent application Ser. No. 13/507,991 a headset carried eye tracker 46 may be employed to measure cognitive performance.

Regardless of the testing modality, it has been found that the use of medications will affect the cognitive function of the brain. It is the correlation between the cognitive function of the brain and the effect of the drug that is administered which gives an indication of whether or not the individual has in fact taken the drug.

For instance, where the cognitive function of the individual establishes a certain baseline, then comparing the cognitive function of the individual at a different time after taking the medication provides an indication of whether or not the patient has in fact taken the drug. If for instance there is no variation in cognitive performance, then it can be deduced that the individual has not taken his or her medication.

On the other hand, if the cognitive function has changed after the administration of the drug, then it can be deduced that the patient has in fact taken the drug.

In summary, it has been found that the cognitive function of a patient on a drug regimen changes with the administration of the drug. It is this measure of the change in cognitive function that enables the ascertaining of compliance with the medication schedule by periodically testing the individual through the utilization of an opto-cognitive testing regime, which may be conveniently administered using portable apparatus available for home use. Note this opto-cognitive testing regime includes any type of opto-cognitive testing regime. In one embodiment the opto-cognitive testing measures the direction of the eye in response to a smoothly moving target on a screen. This then establishes cognitive function, which in turn enables the analysis of the results to ascertain drug compliance.

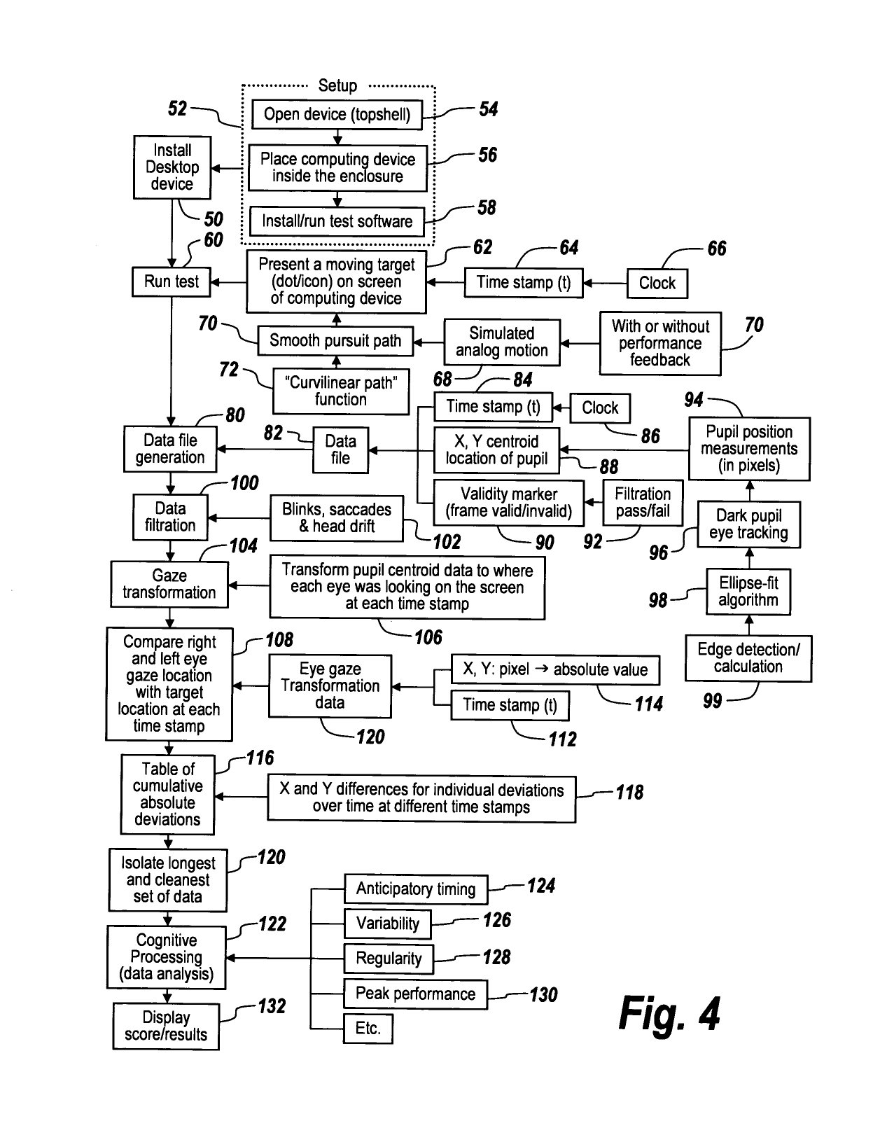

Referring now to FIG. 4, in one embodiment for the precision measurement of cognitive function, as seen at 50 one initially installs a desktop device and sets it up as illustrated at 52 first by opening the top shell of the device is illustrated at 54 and by placing the computing device inside the disclosure as illustrated at 56, whereupon one installs and runs tests software as illustrated at 58. Thereafter a test is run as illustrated at 60 by presenting a moving target which is a dot or icon 62 on a screen of the computing device. This moving target is time stamped at 64 utilizing a clock 66 with simulated analog motion 68 used to generate a smooth pursuit path 70 that is utilized to drive the motion of the icon on the screen. The smooth pursuit path is in one embodiment a curvilinear path 72. Note clock 66 coupled to the simulated analog motion unit 68 as illustrated at 74 with or without a performance feedback.

When the test is run, a data file is generated at 80 from a data file 82 that is in turn time stamped at 84 utilizing a clock 86. Data file 82 stores the X,Y location of the centroid location of the pupil 88. Also stored is a validity marker 90 that a frame is valid or invalid derived from the output of pass/fail and filtration operation 92. The pupil position measurements as illustrated at 94 utilized to derive the X,Y centroid of location of the pupil. These pupil position measurements use pupil eye tracking 96 which incorporates an ellipse-fit algorithm 98 and edge detection calculations 90, thus to accurately determine gaze direction through the X,Y centroid location of the pupil. Having the generated data file 80, one utilizes a data filtration step 100 that eliminates blinks, saccades and head drift as illustrated 102.

Having filtered the data, the next step is gaze transformation 104 in which as illustrated at 106, one transforms pupil centroid data to where each eye is looking on the screen at each time stamp. Gaze direction is ascertained in the traditional manner as described above.

After having transformed the gaze to provide a gaze direction as illustrated, at 108 one compares the left eye and the right eye gaze location with target location at each time stamp. Eye gaze transformation data is available for this process at 110 having been time stamped at 112 and having been derived from an X, Y pixel location transformed into absolute values at 114.

Thereafter a table of cumulative absolute deviations is derived at 116 utilizing X and Y differences for individual deviations over time at different time stamps as illustrated at 118.

Then, the longest and cleanest set of data is isolated at 120 and cognitive processing, namely data analysis, is performed at 122. The cognitive processing includes metrics such as ascertaining anticipatory timing 124, variability 126, regularity 128 and peak performance 130, after which, depending on the metric utilized, the results are displayed at 132 either as a score or some other result representation.

The above processing provides an inordinate amount of processing to filter out outlying data, blinks, saccades, head drift and other environmental factors, such that when gaze direction is calculated all the extraneous effects of noise are eliminated from the gaze direction data. Environmental and head position noise has already been limited by the use of the subject desktop device to eliminate ambient light from getting into the system and to minimize the effect of head movement since the head is clamped to the mask on the desktop device.

What has therefore been described is a desktop system for measuring cognitive performance that is portable and is exceptionally inexpensive and yet provides sufficient accuracy and precision to be useful in clinical drug analysis and specifically for drug taking compliance monitoring.

While the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

What is claimed is:

1. A method for ascertaining drug taking compliance comprising the step of:
utilizing opto-cognitive scanning in order to detect patient drug taking compliance by monitoring short term effects of not taking a prescribed medicine.

2. The method of claim 1, wherein the opto-cognitive scanning technique includes the use of an eye tracking unit and wherein the patient is tested by the eye tracking unit at an interval after the prescribed time for taking the medication.

3. The method of claim 2, wherein the opto-cognitive scanning technique includes static smooth pursuit eye movement measurements.

4. The method of claim 2, wherein the opto-cognitive scanning technique includes a hybrid smooth pursuit eye movement test.

5. The method of claim 1, wherein the opto-cognitive scanning technique is rigorous enough to be useful in the measurement of whether the patient is taking his medicine.

6. The method of claim 5, wherein the opto-cognitive scanning technique precludes faking of the taking of the medicine.

7. The method of claim 6, wherein the opto-cognitive scanning technique has a sufficiently high quantitative resolution to preclude faking the results.

8. The method of claim 1, wherein the opto-cognitive scanning technique utilizes a portable eye-tracking device.

9. The method of claim 8, wherein the portable eye tracking device includes a desktop unit into which an individual peers to look at an internally carried screen on which a moving target proceeds along a smooth pursuit path.

10. The method of claim 9, wherein the desktop unit includes a processor for the analysis of cognitive function relating to the eye tracking of the patient as he peers into the desktop unit.

11. The method of claim 1, wherein the opto-cognitive scanning utilizes an eye tracking metric that includes one of anticipatory timing, variability, regularity and peak performance.

12. The method of claim 8, wherein the portable eye tracking device includes a tablet, which displays a target moving along a smooth pursuit path.

13. The method of claim 12, wherein the patient tracks the moving target utilizing a finger or extension thereof that points to the target as it moves.

14. The method of claim 8, wherein the portable eye tracking device includes a helmet having a screen disposed in front of the patient's eyes when the helmet is in place on the patient and wherein the screen carries a target moving along a smooth pursuit path and further includes a gaze tracking module within the helmet for ascertaining gaze direction as the patient tracks the moving target.

15. Apparatus for ascertaining drug taking compliance comprising:
an opto-cognitive scanning unit adapted to determine cognitive ability of a patient, said opto-cognitive scanning unit being used after a patient's taking a prescribed medicine for ascertaining any change in cognitive performance such that a change in cognitive performance indicates taking of a prescribed medicine.

16. The system of claim 15, wherein said opto-cognitive scanning unit includes an eye tracking unit.

17. The system of claim 16, wherein said eye tracking unit utilizes smooth pursuit eye moving measurements.

18. The system of claim 15, wherein said opto-cognitive scanning unit includes a portable eye tracking device.

19. The system of claim 16, wherein said eye tracking unit includes a desktop unit into which an individual peers to look at an internally carried screen on which a moving target proceeds along a smooth pursuit path.

20. The system of claim 15, wherein said opto-cognitive scanning unit utilizes an eye tracking metric that includes one of anticipatory timing, variability, regularity and peak performance.

21. A system for ascertaining drug taking compliance comprising:
utilizing a tablet that displays a target moving along a smooth pursuit path for ascertaining patient drug compliance by monitoring short term cognitive performance effects.

22. The system of claim 21, wherein cognitive performance is detected by having the patient track a moving target on the tablet screen utilizing a finger or extension thereof that points to the target as it moves.
